(19) United States
(12) Patent Application Publication
(10) Pub. No.: US 2005/0102027 A1
(43) Pub. Date: May 12, 2005

(54) Shims, particularly for laterally placed artificial disc replacements (ADRS)

(76) Inventor: Bret A. Ferree, Cincinnati, OH (US)

Correspondence Address: Gifford, Krass, Groh, Sprinkle,;Anderson & Citkowski, P.C., Suite 400, 280 N. Old Woodward Ave., Birmingham, MI, 48009-5394, US

(21) Appl. No.: 10/987,914

(22) Filed: Nov. 12, 2004

Related U.S. Application Data
(60) Provisional application No. 60/519,372, filed on Nov. 12, 2003.

(51) Int. Cl. A61F 2/44

(52) U.S. Cl. 623/17.11; 623/17.15

(57) ABSTRACT

Shims are placed between the lateral edge of the vertebra and the plate of a laterally placed ADR. The shims assist in the placement of the ADR center of rotation (COR) in the mid-sagittal line. The shims also customize the fit between the lateral surface of the vertebral body and the plate portion of the ADR, since the lateral surface of a vertebral body often has a central concavity. The modular shims also help reduce ADR inventory by facilitating customization of standard ADRs.

### REFERENCE TO RELATED APPLICATIONS

This application claims priority from U.S. Provisional Patent Application Ser. No. 60/519,372, filed Nov. 12, 2003, the entire content of which are incorporated herein by reference.

### FIELD OF THE INVENTION

This invention relates generally to artificial disc replacements (ADRs) and, in particular, to shims applicable to laterally placed ADRs.

### BACKGROUND OF THE INVENTION

Many spinal conditions, including degenerative disc disease, can be treated by spinal fusion or through artificial disc replacement (ADR). ADR has several advantages over spinal fusion. The most important advantage of ADR is the preservation of spinal motion. Spinal fusion eliminates motion across the fused segments of the spine. Consequently, the discs adjacent to the fused level are subjected to increased stress. The increased stress increases the changes of future surgery to treat the degeneration of the discs adjacent to the fusion. However, motion through an ADR also allows motion through the facet joints. Motion across arthritic facet joints could lead to pain following ADR. Some surgeons believe patients with degenerative disease and arthritis of the facet joints are not candidates for ADR.

Current ADR designs do not attempt to limit the pressure across the facet joints or facet joint motion. Indeed, prior art ADRs generally do not restrict motion. For example, some ADR designs place bags of hydrogel into the disc space which do not limit motion in any direction. In fact, ADRs of this kind may not, by themselves, provide sufficient distraction across the disc space. ADR designs with metal plates and polyethylene spacers may restrict translation but they do not limit the other motions mentioned above. The articular surface of the poly spacer is generally convex in all directions. Some ADR designs limit motion translation by attaching the ADR halves at a hinge.

One of the most important features of an artificial disc replacement (ADR) is its ability to replicate the kinematics of a natural disc. ADRs that replicate the kinematics of a normal disc are less likely to transfer additional forces above and below the replaced disc. In addition, ADRs with natural kinematics are less likely to stress the facet joints and the annulus fibrosus (AF) at the level of the disc replacement. Replicating the movements of the natural disc also decreases the risk of separation of the ADR from the vertebrae above and below the ADR.

Compared to spinal fusion and other techniques, prosthetic artificial disc replacements (ADRs) offers many advantages. The prosthetic disc attempts to eliminate a patient's pain while preserving the disc's function. Current prosthetic disc implants either replace the nucleus or the nucleus and the annulus. Current procedures typically remove the degenerated disc component to allow room for the prosthetic component.

Laterally placed ADRs have become more popular, particularly in conjunction with minimally invasive surgical procedures. To ensure proper articulation, however, the center of rotation (COR) should be placed in the mid-sagittal plane. Therefore, a laterally placed ADR must be inserted the proper distance into the disc space to place the COR of the ADR in the mid-sagittal line. Thus, the need remains for further improvements in the way in which prosthetic components are incorporated into the disc space, since the prosthesis may be subjected to 100,000,000 compression cycles over the life of the implant.

### SUMMARY OF THE INVENTION

To address problems associated with the existing art, this invention uses shims between the lateral edge of the vertebra and the plate of a laterally placed ADR. The shims assist in the placement of the ADR center of rotation (COR) in the mid-sagittal line. The shims also customize the fit between the lateral surface of the vertebral body and the plate portion of the ADR, since the lateral surface of a vertebral body often has a central concavity. The modular shims also help reduce ADR inventory by facilitating customization of standard ADRs.

Shims according to the invention could be made of any suitable material, including ceramics, metals such as titanium or chrome-cobalt, or polymers, including in-situ curing polymers. The shims would be available in various shim sizes and shapes.

### BRIEF DESCRIPTION OF THE DRAWINGS

FIG. 1 is a view of the anterior surface of the spine, a laterally placed ADR, and shims according to the invention;

FIG. 2 is a view of the front of shims in various sizes;

FIG. 3 is a view of the front shims in various shapes;

FIG. 4 is an anterior view of the shim drawn in FIG. 2; and

FIG. 5 is an anterior view of the spine and an exploded anterior view of the invention.

### DETAILED DESCRIPTION OF THE INVENTION

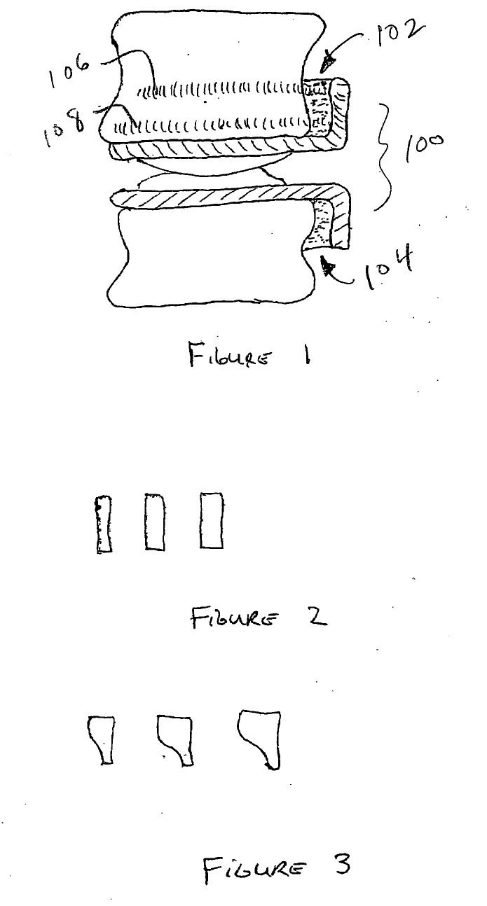

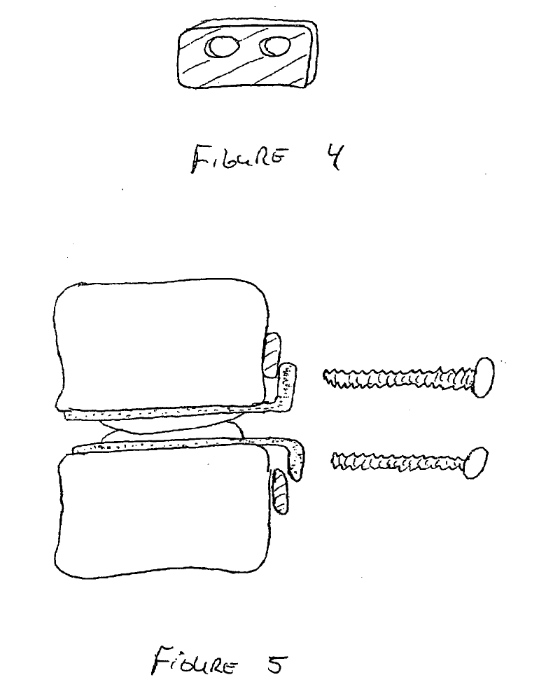

FIG. 1 is a view of the anterior surface of the spine, a laterally placed ADR 100, and shims 102, 104. The trajectory of the screws 106, 108 is illustrated in the upper vertebra. Note that although shims are shown in conjunction with both the upper and lower vertebra, only a single shim may be indicated. FIG. 2 is a view of the front of shims in various sizes. FIG. 3 is a view of the front shims in various shapes. FIG. 4 is an anterior view of the shim drawn in FIG. 2.

FIG. 5 is an anterior view of the spine and an exploded anterior view of the invention. Although the drawings illustrate the use of screws to hold the shims between the ADR and the vertebrae, the invention may include other fastening mechanisms to hold the shims between the ADR and the vertebrae. Alternative embodiments may include the use of shims or ADR components with spring properties, shape-memory properties, plastic deformation, etc. The shims may also be placed between a plate-like component of an ADR and the anterior portion of the spine.

1. Apparatus for use in conjunction with a laterally placed artificial disc replacement (ADR) having a plate with a surface facing the lateral edge of a vertebral body, comprising:
a shim adapted for placement between the plate and the lateral edge of the vertebral body.

2. The apparatus of claim 1, wherein the shim places the center of rotation (COR) of the ADR substantially in the mid-sagittal line.

3. The apparatus of claim 1, including a plurality of differently sized shims to accommodate different plate-vertebrae spacings.

4. The apparatus of claim 1, including a plurality of differently shaped shims to accommodate various lateral edge configurations.
